(19) United States
(12) Patent Application Publication
(10) Pub. No.: US 2005/0085443 A1
(43) Pub. Date: Apr. 21, 2005

(54) Composition and method for treating bezoar and trichobezoar

(76) Inventors: Pavinee Chinachoti, Topeka, KS (US); Luis Jose Montelongo, Lawrence, KS (US)

Correspondence Address: James E. Davis;Harness, Dickey & Pierce, P.L.C., Suite 400, 7700 Bonhomme, St. Louis, MO, 63105, US

(21) Appl. No.: 10/943,694

(22) Filed: Sep. 17, 2004

Related U.S. Application Data
(60) Provisional application No. 60/504,644, filed on Sep. 19, 2003.

(51) Int. Cl. A61K 31/722

(52) U.S. Cl. 514/55

(57) ABSTRACT

This invention is directed generally to methods and compositions (including foods, supplements, treats, toys, etc.) which are useful in preventing or reducing the formation of bezoar or trichobezoar in mammals.

### PRIORITY CLAIM TO RELATED PATENT APPLICATION

This patent claims priority to U.S. Provisional Patent Application Ser. No. 60/504,644 (filed Sep. 19, 2003). The entire text of U.S. Provisional Patent Application Ser. No. 60/504,644 is incorporated herein by reference.

### FIELD OF THE INVENTION

The present invention relates to compositions and methods for preventing gastrointestinal bezoar formation and for reducing the size of a gastrointestinal bezoar in humans and non-human animals.p

### BACKGROUND OF THE INVENTION

Bezoars are any of several types of solid or semi-solid masses of indigestible material found in the stomach of humans and many animals including cattle, cats, rats, rabbits, and non-primates. Bezoars are commonly referred to as “hairballs” and the terms will be used interchangeably throughout this application. Trichobezoars contain a mesh of ingested hair. Trichobezoars are known to entrap undigested dietary fat in the hair mesh. While the incidence of bezoars is generally very low in humans, a somewhat greater risk exists among mentally retarded or emotionally disturbed children, particularly humans who habitually pluck their hair (trichotillomania) and/or eat their hair (trichopagia).

Animals that constantly groom themselves, such as cats and rabbits, regularly ingest large quantities of hair. Normally, the ingested hair passes through the gastrointestinal tract of the animal and is excreted in the feces. However, the ingestion of large quantities of hair may cause the hair to accumulate in the stomach and form a hairball or trichobezoar. A hairball is typically composed of hair, mucous, water, food particles and mineral salts. A hairball can be harmful to the animal if it impedes the normal digestive process by blocking the pylorus and preventing the passage of digesta down the gastrointestinal tract. More frequently, hairballs are nothing more than a nuisance to the animal and its owner. For example, the cat will attempt to eliminate the hairball from the stomach through the vomiting reflex. A hairball may produce constipation and defecation difficulties if it passes from the stomach and becomes lodged in the lower bowel.

### SUMMARY OF THE INVENTION

This invention is directed to methods and compositions for preventing gastrointestinal bezoar formation and for reducing the size of a gastrointestinal bezoar in mammals. It is contemplated that such methods and compositions may be suitable for use in humans and non-human animals.

Briefly, therefore, this invention is directed, in part, to a composition, such as, for example, a food, nutritional supplement, treat, or toy. The composition comprises at least about 0.005% by weight of a biopolymer selected from the group consisting of chitin, chitosan, chitin derivatives and mixtures thereof.

In one contemplated embodiment, the composition comprises from about 0.35% to about 1.0% by weight of a biopolymer selected from the group consisting of chitin, chitosan, chitin derivatives and mixtures thereof.

In another contemplated embodiment, the composition comprises a biopolymer selected from the group consisting of chitin, chitosan, chitin derivatives and mixtures thereof; and from about 0% to about 5% by weight of an antacid or other alkalizing agent.

In another contemplated embodiment, the composition comprises chitin as a biopolymer.

In another contemplated embodiment, the composition comprises chitosan as a biopolymer.

In still another contemplated embodiment, the composition comprises a mixture of chitin and chitosan.

This invention also is directed to an animal treat that comprises a biopolymer selected from the group consisting of chitin, chitosan, chitin derivatives and mixtures thereof.

This invention also is directed to a nutritional supplement that comprises a biopolymer selected from the group consisting of chitin, chitosan, chitin derivatives and mixtures thereof. Preferably, the nutritional supplement is manufactured for consumption by an animal selected from the group consisting of a cat, a dog and a rabbit.

This invention also is directed to an animal toy that comprises a biopolymer selected from the group consisting of chitin, chitosan, chitin derivatives and mixtures thereof.

This invention also is directed to method for using such compositions, nutritional supplements, treats and toys to aid in preventing or reducing the formation of bezoar or trichobezoar in mammals.

The invention also is directed to methods for using such compositions, nutritional supplements, treats and toys to aid weight loss or reduce weight gain in an animal selected from the group consisting of a cat and a dog.

Further benefits of Applicants' invention will be apparent to one skilled in the art from reading this patent.

### BRIEF DESCRIPTION OF THE DRAWINGS

FIG. 1 is a graph demonstrating the average gelling ability of Chitosan 1A as studied in Example 1.

FIG. 2 is a graph demonstrating the average gelling ability of Chitosan 1B as studied in Example 1.

FIG. 3 is a graph demonstrating the average gelling ability of Chitosan 1C as studied in Example 1.

### DETAILED DESCRIPTION OF THE PREFERRED EMBODIMENTS

This detailed description of preferred embodiments is intended only to acquaint others skilled in the art with Applicants' invention, its principles, and its practical application so that others skilled in the art may adapt and apply the invention in its numerous forms, as they may be best suited to the requirements of a particular use. This detailed description and its specific examples, while indicating preferred embodiments of this invention, are intended for purposes of illustration only. This invention, therefore, is not limited to the preferred embodiments described in this specification, and may be variously modified.

It has been found in accordance with this invention that biopolymers selected from the group consisting of chitin, chitosan, chitin derivatives and mixtures thereof can be useful in preventing or reducing the occurrence of bezoars in mammals, particularly in cats. Without being held to a particular theory, Applicants believe that the beneficial effects of the present invention may result from gelation of the biopolymer in the gastrointestinal tract. Specifically, it is believed that dissolved biopolymer which is present in solution under acidic conditions of the stomach undergoes gelation as it passes to higher pH conditions in the intestine. Thus, gelled biopolymer is available in the intestine to entrap and/or lubricate the hairball, thereby allowing passage of the encapsulated hairball through the gastrointestinal tract.

Applicants have further discovered in accordance with the present invention that biopolymers selected from the group consisting of chitin, chitosan, chitin derivatives and mixtures thereof also can be useful in binding or trapping fats in animals, particularly in cats and dogs. Without being held to a particular theory, Applicants believe that biopolymer gels formed in the intestine as described above are also effective in absorbing and/or encapsulating fats. Thus, gelled biopolymer absorbs and/or encapsulates fats in the intestine to allow passage of the absorbed fat through the gastrointestinal tract, thereby aiding in weight loss or reduced weight gain.

Suitable biopolymers for use in the present invention may generally comprise linear polysaccharides such as chitin, chitosan, chitin derivatives and mixtures thereof. Chitin is an abundant biopolymer that is found in the exoskeletons of crustaceans (crab and shrimp) and in the cell walls of fungi and insects. Chitin is a cationic amino polysaccharide consisting of (1-4)-linked 2-acetamido-2-deoxy-β-D-glucopyranose. Chitosan is a collective name for the family of de-acetylated chitins. Thus, chitosan consists of both chitin monomers and chitosan monomers (i.e., 2-amino-2-deoxy-β-D-glucopyranose). Chitin, chitosan and chitin derivatives are further described in Tharanathan et al., Crit. Reviews in Food Sci. & Nutrition, 43(1), pp. 61-87 (2003), which is incorporated herein by reference.

Chitosan and chitin derivatives are often described according to the degree of de-acetylation within the polysaccharide. Accordingly, Applicants have found that all chitosans demonstrate sufficient gelling properties such that the methods and compositions of the present invention are viable with chitosans having all degrees of de-acetylation. However, without being held to a particular theory, results to date further suggest that chitosans with a higher degree of de-acetylation (e.g., at least about 50%, at least about 70% or at least about 90% de-acetylation) produce firmer gels which may be preferred for some applications.

Examples of commercially available chitosans or chitin derivatives suitable for use in the present invention may include Chitosan—Tasteless/Odorless, High Deacetylation Chitosan, LipoSan Ultra and ChitoClea® commercially available from Premix (Iceland); and “Ground Chitosan” or BETASANN commercially available from Arkion Life Sciences, LLC of Wilmington, De.

It is contemplated that the compositions and methods of this invention may be useful for a variety of mammals, including humans and non-human mammals such as non-human primates (e.g., monkeys, chimpanzees, etc.), companion animals (e.g., dogs, cats, rabbits, equine, etc.), farm animals (e.g., goats, sheep, swine, bovine, etc.), laboratory animals (e.g., mice, rats, etc.), and wild and zoo animals (e.g., wolves, bears, deer, etc.).

In some embodiments of this invention, the animal is a cat.

In other embodiments of this invention, the animal is a dog.

In other embodiments of this invention, the animal is a rabbit.

This invention contemplates a variety of compositions containing biopolymers. Contemplated compositions include, for example, foods, supplements, treats, snacks and toys (typically chewable and consumable toys). Additionally, the chitin, chitosan or chitin derivatives can be administered in oral unit dosage form such as a tablet, capsule, pill or the like.

Depending on the application, the biopolymer preferably is present in the composition in an amount which is therapeutically effective for either (a) preventing or treating bezoar or trichobezoar in a mammal or (b) aiding weight loss or reducing weight gain in a mammal. For example, the concentration of biopolymer in a composition of this invention is at least about 0.005% by weight of the composition (or from about 0.1% to about 30%, or from about 0.1% to about 10% or from about 0.2% to about 5%, or from about 0.35% to about 1.0% based on the weight of the composition). It is important to note that when the composition is a food, it is contemplated that the biopolymer may comprise up to about 10% by weight of the composition. However, when the composition is a treat, toy or nutritional supplement, it is contemplated that the biopolymer may comprise as much as about 30% by weight of the composition.

In another embodiment, the composition comprises a biopolymer and an antacid or other alkalizing agent. For example, without being held to a particular theory, Applicants have discovered that elevating the pH of the local stomach environment when administering the biopolymer further enhances gel formation of the biopolymer. In particular, increasing the local pH of the stomach environment is believed to promote early biopolymer gel formation in the stomach which may further facilitate the passage of hairballs or absorbed fats to the small intestine. Because the gel formed in the stomach is preserved under conditions in the intestine, passage of the hairball or absorbed fats is further facilitated through the GI tract.

Suitable antacids or alkalizing agents for use in the compositions of the invention may include any antacid known to those skilled in the art which can be used as a food ingredient or food additive. Particular contemplated antacids include phosphate salts (e.g., sodium phosphate monobasic, sodium phosphate dibasic, sodium acid pyrophosphate, sodium tetrapyrophosphate), zinc acetate or mixtures thereof. The antacid or alkalizing agent is preferably present in the composition in an amount which is therapeutically effective to elevate the pH of the local stomach environment when the composition is administered to a mammal. For example, when administered in a composition of the present invention, the concentration of antacid or alkalizing agent is from about 0% to about 5% by weight of the composition (or from about 0.1% to about 1%, or from about 0.2% to about 0.75%, or about 0.5% based on the weight of the composition).

Typically, the biopolymer and other ingredients of the composition are present at concentrations that do not impart, when combined, an odor or flavor that causes the intended animal to perceive the composition to be unacceptable for consumption. In many instances, a desirable odor and flavor can be achieved using aroma or flavor enhancers.

The biopolymer and other ingredients preferably are present at concentrations that are not deleterious to the intended animal's health. Thus, for example, the biopolymer and other ingredients preferably are present at concentrations that do not cause undesirable effects on digestion, particularly long term undesirable effects on digestion, such as undesirable effects lasting several days or longer. Undesirable effects on digestion may include, for example, constipation or diarrhea.

In one embodiment, the composition is a food supplement comprising a biopolymer selected from the group consisting of chitin, chitosan, chitin derivatives and mixtures thereof. Supplements include, for example, a feed or pet food used with another feed or pet food to improve the nutritive balance or performance of the total. Contemplated supplements include compositions that are fed undiluted as a supplement to other feeds or pet foods, offered free choice with other parts of an animal's ration that are separately available, or diluted and mixed with an animal's regular feed or pet food to produce a complete feed or pet food. The AAFCO, for example, provides a discussion relating to supplements in the American Feed Control Officials, Incorp. Official Publication, p.220 (2003). Supplements may be in various forms including, for example, powders, liquids, syrups, pills, encapsulated compositions, etc.

In another embodiment, the composition is a treat comprising a biopolymer selected from the group consisting of chitin, chitosan, chitin derivatives and mixtures thereof. Treats include, for example, compositions that are given to an animal to entice the animal to eat during a non-meal time. Contemplated treats for canines include, for example, dog bones. Treats may be nutritional, wherein the composition comprises one or more nutrients, and may, for example, have a composition as described above for food. Non-nutritional treats encompass any other treats that are non-toxic. The biopolymer can be coated onto the treat, incorporated into the treat, or both. It is important to note that when the composition of the invention is a treat, the concentration of biopolymer in the treat may be as high as 30% by weight.

In another embodiment, the composition is a toy comprising a biopolymer selected from the group consisting of chitin, chitosan, chitin derivatives and mixtures thereof. Toys include, for example, chewable toys. Contemplated toys for dogs include, for example, artificial bones. The biopolymer can form a coating on the surface of the toy or on the surface of a component of the toy, be incorporated partially or fully throughout the toy, or both. In a contemplated embodiment, the biopolymer is orally accessible by the intended user. There a wide range of suitable toys currently marketed. See, e.g.,, U.S. Pat. No. 5,339,771 (and references disclosed in U.S. Pat. No. 5,339,771). See also, e.g., U.S. Pat. No. 5,419,283 (and references disclosed in U.S. Pat. No. 5,419,283). It should be recognized that this invention contemplates both partially consumable toys (e.g., toys comprising plastic components) and fully consumable toys (e.g., rawhides and various artificial bones). It should be further recognized that this invention contemplates toys for both human and non-human use, particularly for companion, farm, and zoo animal use, and particularly for dog, cat, or rabbit use.

In preparing a composition of the present invention, the components of the composition are adjusted so that the biopolymer is present in the composition at a concentration of at least about 0.005% (or from about 0.1% to about 30%, or from about 0.1% to about 10%, or from about 0.2% to about 5%, or from about 0.35% to about 1%) based on the dry content of the composition. The biopolymer may, for example, be incorporated into the composition during the processing of the formulation, such as during and/or after mixing of other components of the composition. Distribution of these components into the composition can be accomplished by conventional means. It is important to note that when the composition is a food, it is contemplated that the biopolymer may comprise up to about 10% by weight of the composition. However, when the composition is a treat, toy or nutritional supplement, it is contemplated that the biopolymer may comprise as much as about 30% by weight of the composition.

Compositions of the present invention (particularly foods) can be prepared in a canned or wet form using conventional pet food processes. In one contemplated embodiment, ground animal (e.g., mammal, poultry, and/or fish) proteinaceous tissues are mixed with the other ingredients, including animal fats and vegetable oils, cereal grains, other nutritionally balancing ingredients, special purpose additives (e.g., vitamin and mineral mixtures, inorganic salts, cellulose and beet pulp, bulking agents, and the like); and water that sufficient for processing is also added. These ingredients preferably are mixed in a vessel suitable for heating while blending the components. Heating of the mixture may be effected using any suitable manner, such as, for example, by direct steam injection or by using a vessel fitted with a heat exchanger. Following the addition of the last ingredient, the mixture is heated to a temperature range of from about 50° F. to about 212° F. Temperatures outside this range are acceptable, but may be commercially impractical without use of other processing aids. When heated to the appropriate temperature, the material will typically be in the form of a thick liquid. The thick liquid is filled into suitable containers such as cans, jars, pouches or the like. A lid is applied, and the container is hermetically sealed. The sealed containers are then placed into conventional equipment designed to sterilize the contents. This is usually accomplished by heating to temperatures of greater than about 230° F. for an appropriate time, which is dependent on, for example, the temperature used and the composition. Products may also be prepared by an aseptic process wherein the contents are heated to commercial sterility before being packaged in sterilized containers.

Compositions of the present invention (particularly foods) can be prepared in a dry form using conventional processes. In one contemplated embodiment, dry ingredients, including, for example, animal protein sources, plant protein sources, grains, etc., are ground and mixed together. Moist or liquid ingredients, including fats, oils, animal protein sources, water, etc., are then added to and mixed with the dry mix. The mixture is then processed into kibbles or similar dry pieces. Kibble is often formed using an extrusion process in which the mixture of dry and wet ingredients is subjected to mechanical work at a high pressure and temperature, and forced through small openings and cut off into kibble by a rotating knife. The wet kibble is then dried and optionally coated with one or more topical coatings which may include, for example, flavors, fats, oils, powders, and the like. Kibble also can be made from the dough using a baking process, rather than extrusion, wherein the dough is placed into a mold before dry-heat processing. Kibble also can be made from a food matrix undergoing pelletization. It is important to note that the biopolymer may be incorporated into the food composition by adding the biopolymer to the above-described mixtures before extrusion or by coating the extruded kibble or pellets with the biopolymer as a topical coating.

Treats of the present invention can be prepared by, for example, an extrusion or baking process similar to those described above for dry food. Other processes also may be used to either coat the biopolymer on the exterior of existing treat forms, or inject it into an existing treat form.

Animal toys of the present invention are typically prepared by coating any existing toy with the biopolymer.

### EXAMPLES

The following examples are merely illustrative, and are not intended to limit this disclosure in any way.

### Example 1

This experiment was conducted to characterize gel formation by various chitosan solutions. The experiment was conducted using 1.0% chitosan solutions prepared from three commercially available chitosans. The commercially available chitosans included:

Chitosan 1A—“Chitosan Tasteless/Odorless” from Premix (Iceland)

Chitosan 1B—“High Deacetylation Chitosan” (indicated as having no less than 78% deacetylation) from Premix (Iceland)

Chitosan 1C—ChitoClear® (indicated as having more than 90% deacetylation) from Premix (Iceland).

1.0% chitosan solutions were prepared by mixing the chitosan (1.0 g) with 0.16N HCl (75 ml) for 15 minutes. An additional amount of 0.16N HCl was added to make a 100 ml solution and the solution was mixed for an additional 2 hours, 45 minutes. After the mixing period, the pH was measured and the chitosan solution was stored under refrigeration.

Gelling was measured by placing nine samples of the chitosan solution (10 g each) into centrifuge tubes. 1N or 0.1N NaOH or 1N or 0.1 N HCl was added to each tube to adjust the pH as follows:

Tube 1—pH 2.0

Tube 2—pH 3.0

Tube 3—pH 4.0

Tube 4—pH 5.0

Tube 5—pH 5.5

Tube 6—pH 5.75

Tube 7—pH 6.0

Tube 8—pH 7.0

Tube 9—pH 8.0

Each of the tubes were then centrifuged at 3200 rpm for 20 minutes at room temperature. Gel formation was quantified by separating the gel (precipitate) using a filtration system and catching any precipitate on pre-weighed Whatman paper. The precipitates were then weighed after drying overnight.

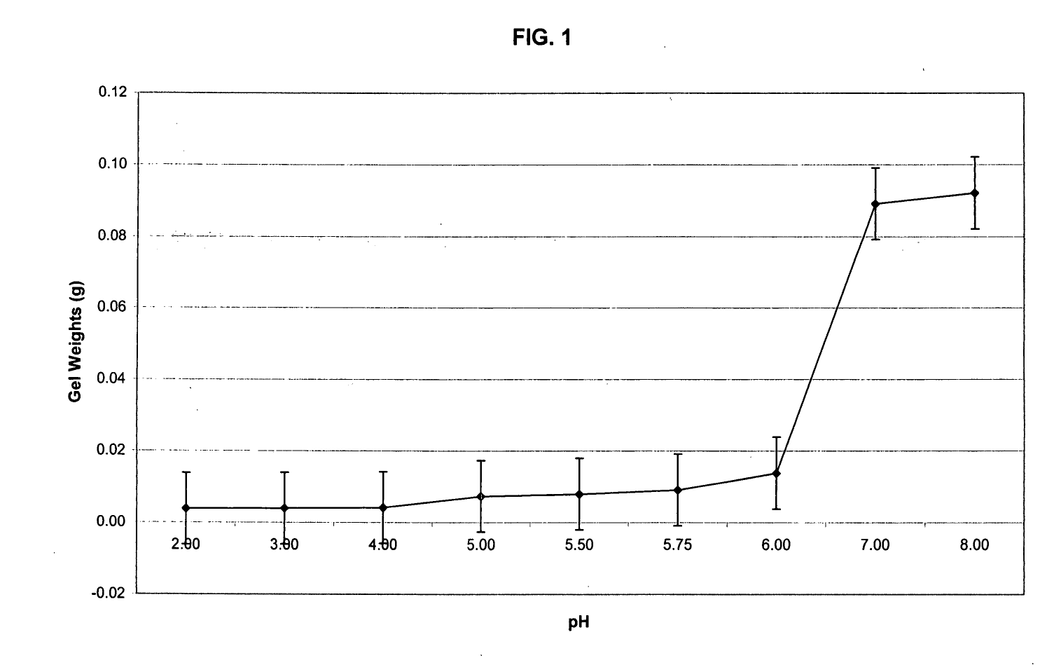

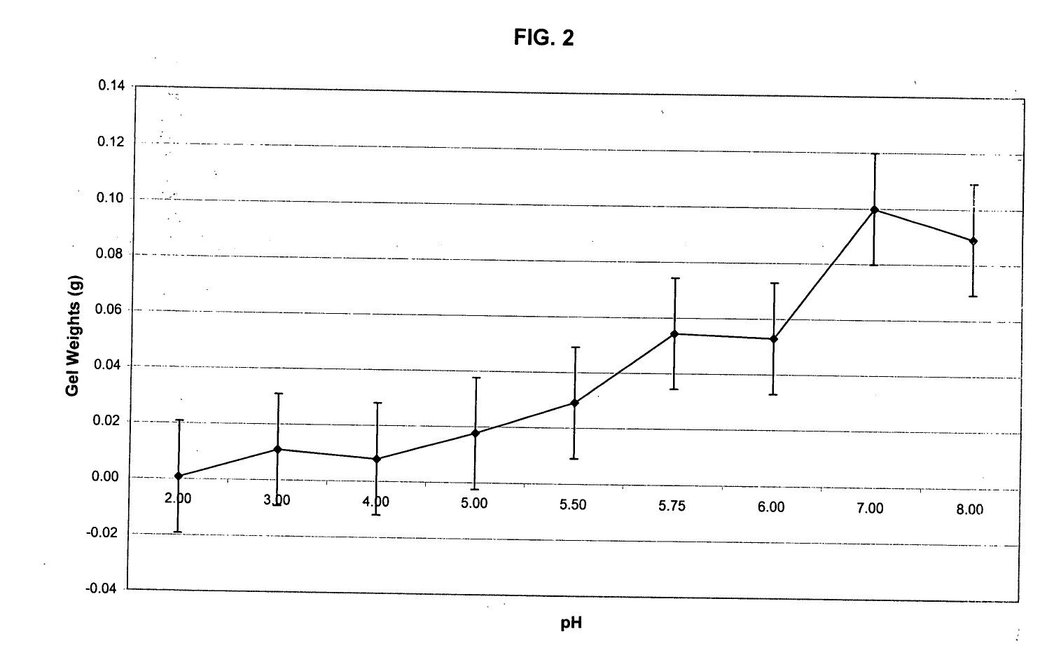

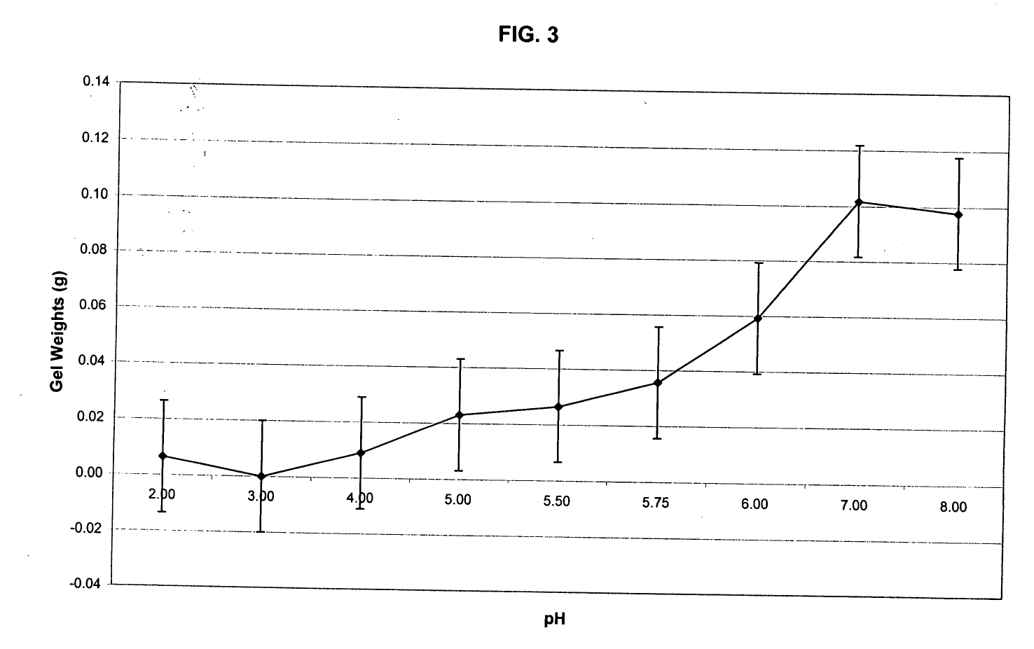

FIGS. 1 to 3 show the results of gel weight versus pH for the experiment. Results indicated that very little gelling occurred with Chitosan 1A below pH 6.0 and that maximum gelling occurred at pH 7.0 and 8.0 (see FIG. 1). For Chitosan 1B, gelling occurred as low as pH 5.5 with increasing gel formation as pH increased (see FIG. 2). Chitosan 1C showed minimal gelling at pH 5.5 and 5.75 but significant gelling at pH 6.0 and above (see FIG. 3).

### Example 2

This experiment comprised an in vitro study of the effect of chitosan on hairball formation. The experiment was conducted using a 1.0% chitosan solution at pH 1.0.

Solutions of 1.0% chitosan were prepared by mixing chitosan (1.0 g) with 0.16N HCl (75 ml) for 15 minutes. An additional amount of 0.16N HCl was added to make a 100 ml solution and the solution was mixed for an additional 2 hours, 45 minutes. After the mixing period, the pH was measured and the chitosan solutions were stored under refrigeration.

The mixtures were evaluated by adding the chitosan solutions (10 g) to centrifuge tubes and adjusting the pH to about 1.0. A feline hairball (1-3 g) was added to each centrifuge tube and pH was measured. The tubes were closed and inverted for 1 minute before setting them in a water bath at 37° C. and 50-120 rpm for 2 hours.

The hairballs were next evaluated under intestine conditions by adding pH 10.5 carbonate/bicarbonate buffer (8.5 ml) to each tube. The tubes were closed and inverted for 1 minute before standing. After one hour, pH was measured and the tubes were adjusted to pH 5.7, 6.0, 6.5, 7.0, 7.5 and 8.0 before being placed in a water bath at 37° C. without shaking for 2 hours.

Results indicated that the chitosan interacted with the hairball, and during the addition of the carbonate/bicarbonate buffer, a gel was formed around the hairball and the hairball became dispersed.

### Example 3

This experiment comprised an in vitro study of the effect of chitosan and sodium phosphate dibasic in combination with various amounts of zinc acetate on hairballs at pH 3.0. The purpose of this study was to determine the effect of adding sodium phosphate dibasic and/or zinc acetate on chitosan gelling. The experiment was conducted using a 0.5% chitosan solution at pH 3.

A solution of 0.5% chitosan was prepared by mixing chitosan (0.5 g) with 0.16N HCl (75 ml) for 15 minutes. An additional amount of 0.16N HCl was added to make a 100 ml solution and the solution was mixed for an additional 2 hours, 45 minutes. After the mixing period, the pH was measured and the chitosan was stored under refrigeration.

The mixtures were evaluated under stomach conditions by adjusting the chitosan solution to pH 3.0 with sodium hydroxide. The chitosan solution (10 g) was then added to each of twelve centrifuge tubes. Various tubes then received additional amounts of sodium phosphate dibasic and/or zinc acetate as follows:

Tubes 1 and 1A—no salt added

Tubes 2 and 2A—sodium phosphate dibasic (0.5%)

Tubes 3 and 3A—zinc acetate (0.1%)

Tubes 4 and 4A—sodium phosphate dibasic (0.5%) and zinc acetate (0.10%)

Tubes 5 and 5A—sodium phosphate dibasic (0.5%) and zinc acetate (0.075%)

Tubes 6 and 6A—sodium phosphate dibasic (0.5%) and zinc acetate (0.05%).

pH was measured before and after the salt addition. The tubes were mixed for 5 minutes at high speed using a Roto Shake Ginnie (Scientific Industries, Inc). A feline hairball (1 -3 g) was added to each centrifuge tube and pH was measured again. The tubes were closed and inverted for 1 minute before setting them in a water bath at 37° C. and 120 rpm for 2 hours.

The hairballs were next evaluated under intestine conditions by adding pH 10.5 carbonate/bicarbonate buffer (8.5 ml) to each tube. The tubes were closed and inverted for 1 minute before standing. After one hour, the tubes were placed in a water bath at 37° C. without shaking for 2 hours before measuring final pH.

Results are shown in Table 1 below. The results showed that there was no formation of gel around the hairball in the control sample without the addition of 0.5% sodium phosphate dibasic. Without being held to a particular theory, it is believed that no gel is formed because the pH of the solution remained the same. However, the addition of 0.5% sodium phosphate dibasic changed the pH from 3 to 6.7. This pH increase promoted the development of a gel surrounding the hairball. Such a development is important because the addition of the phosphate salt will allow the formation of a gel under conditions in the stomach, which may facilitate the passage of the hairball from the stomach to the intestine.

After the addition of the carbonate/bicarbonate buffer pH 10.5, the control sample (intestine condition) showed the formation of a soft gel surrounding the hairball and a gel-like precipitation in the liquid. The gel formed because the pH of the solution increased above neutral pH. The sample with the addition of sodium phosphate dibasic (intestine conditions) kept similar gel as with the stomach condition. The pH of the sample was about 10. The formation of the gel with the addition of the phosphate salts in the stomach conditions and the preservation of the gel in the intestine condition may ease the passage of the hairball through the gastrointestinal tract.

Addition of 0.1% zinc acetate increased the pH of the solution to 4.5 as can be observed in Table 1 but the addition did not result in a gel surrounding the hairball and the liquid showed no gel-like precipitation. The addition of a combination of 0.5% sodium phosphate dibasic and 0.1% zinc acetate increased the pH from 3 to 6.4 and showed gel formation on the hairball along with a gel-like precipitate in the liquid. However, the gel was not as visually strong as the one formed with the addition of only 0.5% sodium phosphate dibasic. A similar pH increase of 6.6 was observed with the additions of 0.5% sodium phosphate dibasic and 0.75% zinc acetate and 0.5% sodium phosphate dibasic and 0.50% zinc acetate (Table 1). However, less gel was observed with these treatments. After the addition of carbonate/bicarbonate pH 10.5 buffer (intestine conditions) the pH of the samples increased to about 10 (Table 1). The addition of 0.1% Zinc acetate showed a white film around the hairball, which seemed to be a soft gel. Samples with the addition of 0.5% sodium phosphate dibasic and 0.1, 0.75 and 0.5% zinc acetate respectively showed the formation of gel-like precipitate and no gel.

Based on the observations made, it is concluded that the addition of sodium phosphate dibasic increased the pH of the chitosan solution and formed a very strong gel around the hairball in the first step (stomach conditions), before the addition of the buffer. Thus, the addition of an antacid or other alkalizing agent such as a phosphate salt may sufficiently increase the pH in the local stomach environment to allow the biopolymer to gel around the hairball. Accordingly, early gel formation in the stomach may further facilitate passage of the hairball to the small intestine. Also, the gel formed in the stomach conditions seems to be preserved under conditions in the intestine, further easing the passage of the hairball through the gastrointestinal tract. Therefore, in order to trap and/or encapsulate hairballs in the stomach, delivery of an effective antacid or other suitable alkalizer effective to increase the pH of the local stomach environment may be beneficial.

TABLE 1

| | Hair ball | | | |
|---|---|---|---|---|
| Tube # | wt.(g) | Salt Added to Chitosan | pH stomach | pH intestine |
| 1 | 1.66 | Control (no salt added) | 2.98 | 10.07 |
| 1-A | 1.40 | Control (no salt added) | 2.94 | 10.00 |
| 2 | 1.06 | 0.5% sodium phosphate dibasic | 6.69 | 10.21 |
| 2-A | 1.23 | 0.5% sodium phosphate dibasic | 6.67 | 10.08 |
| 3 | 1.70 | 0.1% zinc acetate | 4.48 | 10.04 |
| 3-A | 1.93 | 0.1% zinc acetate | 4.85 | 10.20 |
| 4 | 1.22 | 0.5% sodium phosphate dibasic + 0.1% | 6.39 | 9.90 |
| | | zinc acetate | | |
| 4-A | 1.36 | 0.5% sodium phosphate dibasic + 0.1% | 6.55 | 10.04 |
| | | zinc acetate | | |
| 5 | 1.38 | 0.5% sodium phosphate dibasic + 0.075% | 6.55 | 10.06 |
| | | zinc acetate | | |
| 5-A | 1.53 | 0.5% sodium phosphate dibasic + 0.075% | 6.64 | 10.02 |
| | | zinc acetate | | |
| 6 | 1.84 | 0.5% sodium phosphate dibasic + 0.05% | 6.57 | 10.13 |
| | | zinc acetate | | |
| 6-A | 2.03 | 0.5% sodium phosphate dibasic + 0.05% | 6.59 | 9.82 |
| | | zinc acetate | | |

### Example 4

This experiment studied the oil absorption properties of various commercially available chitosans. The experiment was conducted on six commercially available chitosans as follows:

Chitosan 4A—“Chitosan Tasteless/Odorless” from Premix (Iceland).

Chitosan 4B—“High Deacetylation Chitosan” from Premix (Iceland).

Chitosan 4C—“LipoSan Ultra” from Premix (Iceland).

Chitosan 4D—ChitoClear® from Premix (Iceland).

Chitosan 4E—“Ground Chitosan” from Arkion Life Sciences, LLC.

Chitosan 4F—BETASANN from Arkion Life Sciences, LLC.

The experiment was conducted by preparing solutions of each chitosan. The solutions were prepared by mixing each chitosan (2 g) with 0.16 N HCl (150 ml) and stirring the mixture for 15 minutes. An additional amount of 0.16N HCl was added to make 200 ml of a 1% chitosan solution. 100 ml of solution was separated and stored as the 15 minute hydration samples while the remaining 100 ml of solution was stirred for an additional 2 hours, 45 minutes. The pH of the solution was then measured before storing the solution under refrigeration.

Each of the chitosan solutions (10 g) were placed in centrifuge tubes (with duplicates). A mixture (10 g) of animal fat and vegetable oil was added to each of the centrifuge tubes except for a control. The centrifuge tubes were closed and mixed using a vortex. The tubes were inverted for a minute before setting in a water bath at 37° C. and 120 rpm for 20 minutes. After 20 minutes, a carbonate/bicarbonate buffer (5 ml, pH 10.5) was added. The tubes were closed and inverted for 1 minute before measuring pH. The tubes were then placed in a water bath at 37° C. and 120 rpm. After 2 hours in the water bath, the tubes were removed. Excess oil supernatant was weighed and recorded. Table 2 shows the results.

TABLE 2

| | Amount of unabsorbed | | Amount of unabsorbed | |
|---|---|---|---|---|
| | fat in chitosan | | fat in chitosan | |
| | with 15 min. hydration | | with 3 hour hydration | |
| Chitosan | Oil released | Oil absorbed | Oil released | Oil absorbed |
| 4A | 5.65 ± 0.19a | 4.35 ± 0.19a | 5.26 ± 0.01a | 4.74 ± 0.01a |
| 4B | 4.29 ± 0.05a | 5.71 ± 0.05a | 3.81 ± 0.06a | 6.19 ± 0.05a |
| 4C | 5.61 ± 0.27a | 4.39 ± 0.27a | 4.87 ± 0.22a | 5.13 ± 0.22a |
| 4D | 3.99 ± 0.16a | 6.01 ± 0.16a | 3.39 ± 0.14a | 6.61 ± 0.14a |
| 4E | 4.35 ± 0.34a | 5.65 ± 0.34a | 4.79 ± 0.32a | 5.21 ± 0.32a |
| 4F | 7.07 ± 0.06a | 2.93 ± 0.06a | 7.17 ± 0.04a | 2.83 ± 0.04a |
| Control | 8.28 ± 0.26b | 1.72 ± 0.26b | 8.28 ± 0.26b | 1.72 ± 0.26b |

** Note:
Indicators “a” and “b” are intended to show comparisons within each column.

The goal of the experiment was to determine which of the six commercial chitosans studied had better fat absorption ability and to determine if hydration time during chitosan solution preparation affected fat absorption. Results indicated samples which were hydrated for three hours held oil better than the samples which were hydrated for only 15 minutes. These results were confirmed with statistical analysis which indicated that significant differences existed between 15 minute samples and 3 hour samples for all chitosans except Chitosan 4F. Each of the chitosans demonstrated oil absorption ability with Chitosans 4B, 4D, 4A and 4E having the best gel structure and oil absorption ability.

All the references cited above are incorporated by reference into this patent.

The words “comprise”, “comprises”, and “comprising” are to be interpreted inclusively rather than exclusively.

The above detailed description of preferred embodiments is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

1. A method for treating or preventing a bezoar or trichobezoar in a mammal, the method comprising administering to the mammal a therapeutically effective amount of a biopolymer selected from the group consisting of chitin, chitosan, chitin derivatives and mixtures thereof.

2. The method as set forth in claim 1, wherein method comprises administering to the mammal a composition comprising at least about 0.005% by weight biopolymer.

3. The method as set forth in claim 1, wherein the method comprises administering to the mammal a composition comprising from about 0.1% to about 10% by weight biopolymer.

4. The method as set forth in claim 1, wherein the method comprises administering to the mammal a composition comprising from about 0.35% to about 1.0% by weight biopolymer.

5. The method as set forth in claim 1, wherein the method further comprises administering an alkalizing agent to the mammal.

6. The method as set forth in claim 5, wherein the alkalizing agent is administered to the mammal in an amount which is therapeutically effective to elevate the pH of the local stomach environment.

7. The method as set forth in claim 5, wherein administration of the alkalizing agent is effective to elevate the pH of the local stomach environment of the mammal above neutral.

8. The method as set forth in claim 5, wherein the alkalizing agent is a phosphate salt.

9. The method as set forth in claim 1, wherein the biopolymer comprises chitin.

10. The method as set forth in claim 1, wherein the biopolymer comprises chitosan.

11. The method as set forth in claim 1, wherein the biopolymer comprises a mixture of chitin and chitosan.

12. The method as set forth in claim 1, wherein the mammal is a human.

13. The method as set forth in claim 1, wherein the mammal is a dog.

14. The method as set forth in claim 1, wherein the mammal is a cat.

15. The method as set forth in claim 1, wherein the mammal is a rabbit.

16. A composition for animal consumption comprising at least about 0.005% by weight of a biopolymer selected from the group of chitin, chitosan, a chitin derivative and combinations thereof.

17. A composition as set forth in claim 16, wherein the biopolymer comprises chitin.

18. A composition as set forth in claim 16, wherein the biopolymer comprises a mixture of chitin and chitosan.

19. A composition as set forth in claim 16, wherein the biopolymer comprises chitosan.

20. A composition as set forth in claim 19, wherein the composition comprises from about 0.1% to about 10% by weight chitosan.

21. A composition according to claim 19, wherein the composition comprises from about 0.35% to about 1.0% by weight chitosan.

22. A composition as set forth in claim 16, wherein the composition further comprises from about 0% to about 5% by weight of an alkalizing agent.

23. A composition as set forth in claim 22, wherein the alkalizing agent is a phosphate salt.

24. An animal treat, wherein the treat comprises a biopolymer selected from the group consisting of chitin, chitosan, chitin derivatives and mixtures thereof.

25. An animal treat according to claim 24, wherein:
the treat comprises from about 0.1% to about 30% biopolymer (based on dry weight of the treat), and
the treat is manufactured for consumption by an animal selected from the group consisting of a cat, a dog and a rabbit.

26. An animal toy, wherein the toy comprises a biopolymer selected from the group consisting of chitin, chitosan, chitin derivatives and mixtures thereof.

27. An animal toy according to claim 26, wherein the toy is manufactured for use by an animal selected from the group consisting of a cat, a dog and a rabbit.

28. A nutritional supplement, wherein the supplement comprises a biopolymer selected from the group consisting of chitin, chitosan, chitin derivatives and mixtures thereof; and
the supplement is manufactured for consumption by an animal selected from the group consisting of a cat, a dog and a rabbit.

29. A method for aiding weight loss or reducing weight gain in an animal selected from the group consisting of a cat and a dog, the method comprising:
administering to the animal a therapeutically effective amount of a biopolymer selected from the group consisting of chitin, chitosan, chitin derivatives and mixtures thereof.

30. The method as set forth in claim 29, wherein the biopolymer is administered to the animal by feeding the animal a composition of claim 16.
